(19) United States
(12) Patent Application Publication
(10) Pub. No.: US 2004/0126804 A1
(43) Pub. Date: Jul. 1, 2004

(54) Homology analysis system, homology analysis method, homology analysis program, and transaction establishment system

(76) Inventors: Takao Shinozawa, Kumagaya-Shi (JP); Tokumasa Horiike, Kiryu-Shi (JP); Kazuo Hamada, Kiryu-Shi (JP); Shigehiko Kanaya, Ikoma-Shi (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/737,120

(22) Filed: Dec. 17, 2003

Related U.S. Application Data
(63) Continuation of application No. PCT/JP01/10217, filed on Nov. 22, 2001.

(30) Foreign Application Priority Data
Jun. 18, 2001 (JP) 2001-183856
Jul. 31, 2001 (JP) 2001-231810

(51) Int. Cl. C12Q 1/68; G06F 19/00; G01N 33/48; G01N 33/50

(52) U.S. Cl. 435/6; 702/20

(57) ABSTRACT

A BLAST analysis unit calculates the homologies between data included in an analysis target data group and the first data group, respectively, and calculates the number of data exhibiting homologies (orthologous gene count) as a first homology value x by using the calculated values. This unit sets n_{A} thresholds E and calculates a first homology value xᵢ for each threshold Eᵢ, and calculates the homologies between data included in the analysis target data group and the second data group, respectively. The unit calculates the number of data exhibiting homologies (orthologous gene count) as a second homology value y. The unit sets n_{B} thresholds E and calculates a second homology value yᵢ for each threshold Eᵢ. A t-test unit determines to which one of the first and second data groups the analysis target data group is similar.

### CROSS-REFERENCE TO RELATED APPLICATIONS

This is a Continuation Application of PCT Application No. PCT/JP01/10217, filed Nov. 22, 2001, which was not published under PCT Article 21 (2) in English.

This application is based upon and claims the benefit of priority from the prior Japanese Patent Applications No. 2001-183856, filed Jun. 18, 2001; and No. 2001-231810, filed Jul. 31, 2001, the entire contents of both of which are incorporated herein by reference.

### BACKGROUND OF THE INVENTION

1. Field of the Invention

The present invention relates to a homology analysis system, homology analysis method, homology A analysis program, and transaction establishment system which analyze the homologies between data groups each including a plurality of data.

2. Description of the Related Art

The most popular method as a conventional method of comparing the similarities between genes is “phylogenetic tree analysis”. This method can analyze interrelationships including the similarities between genes. An analysis of many genes, however, has a drawback in that it requires a long period of time. In addition, in analyzing the overall similarities between individual organisms, e.g., the interrelationships between many genes as masses, there is no guarantee that appropriate genes are always selected. This is because there is no telling that adopted genes really represent the relationship between the corresponding organisms. In addition to the above phylogenetic tree analysis, the BLAST method and FASTA method are typical methods of comparing the homologies/similarities between genes. In this case, the BLAST method is a method of performing an analysis by using a program for selecting genes having similarities with a specific gene from a gene databank.

The BLAST method and FASTA method can select and specify genes by analyzing which genes have how many degrees of similarity in accordance with the degrees of similarity between genes, i.e., how high the similarities are. In these methods, however, an area in which a degree of similarity is set, i.e., a threshold, is fixed. These methods are therefore insufficient in, for example, analyzing which kinds of genes exist in other degrees of similarities.

### BRIEF SUMMARY OF THE INVENTION

It is an object of the present invention to provide a homology analysis system, homology analysis method, homology analysis program, and transaction establishment system which can compare many data with each other quantitatively and accurately.

According to an aspect of the present invention, there is provided a homology analysis system for analyzing whether an analysis target data group is similar to a first data group or a second data group wherein the first and second data groups is different from the analysis target data group, comprising, a first homology value calculation unit calculating a first homology value x representing a homology between data included in the analysis target data group and the first data group respectively, wherein the first homology value calculating unit sets n thresholds E each indicating a determination criterion for the presence/absence of a homology and calculates a first homology value xᵢ (i=1, 2, . . . , n) for each threshold Eᵢ, a second homology value calculation unit calculating a second homology value y representing a homology between data included in the analysis target data group and the second data group respectively, wherein the second homology value calculating unit sets n thresholds E each indicating a determination criterion for the presence/absence of a homology and calculates a second homology value yᵢ (i=1, 2, . . . , n) for each threshold Eᵢ, and homology determination unit determining to which one of the first and second data groups the analysis target data group is similar on the basis of a relationship between the first homology value xᵢ, the second homology value yᵢ, and the number n of thresholds.

According to this arrangement, the homologies, i.e., the similarities, between data groups each including many data can be evaluated altogether. In addition, by using genetic data as data, the homologies between many gene groups can be evaluated altogether. Therefore, there is no need to select representative genes to draw a phylogenetic tree as in the prior art. This makes it possible to understand the characteristics of gene groups more accurately.

For example, a conventional method of checking the relationship between many gene groups is a method of selecting several genes representing each gene group and drawing their phylogenetic tree. According to this method, however, it cannot be determined whether the selected genes are really appropriate as representative genes. In this case, the result greatly varies depending on whether or not the selected genes are appropriate. In contrast to this, according to the BLAST method or FASTA method, homology searches are performed for all genes (ORFs) included in a target data group. According to these methods of counting the number of genes (ORFs) reaching a given threshold, their relationships can be estimated. In these methods, however, if gene groups include several gene groups having different origins, the result greatly varies as the threshold reference is changed.

As in the present invention, therefore, homology searches are performed by using various thresholds. The present invention then uses a method of plotting the numbers of genes (ORFs) exhibiting homologies with the respective thresholds and estimating the relationships between the gene groups. This makes it possible to obtain very stable homology evaluation results without considering the appropriateness of thresholds.

Currently, after the elucidation of the human genome, there has been a big trend toward placing great importance on selecting/specifying genes that cause diseases. A gene candidate is produced as a DNA fragment from an actual human DNA sample by the PCR gene amplification method. A protein synthesized by the gene is actually produced by an expression system such as Escherichia coli. The activity of the protein is then observed or an antibody for the protein is produced. The antibody can be effectively used for a disease detection method or the like by being actually applied to a pathological section or the like. In order to specify a gene that causes a disease, a morbid area of the tissue of the patient is specified or the genes of a healthy person are analyzed. This can discriminate a person who is vulnerable to the disease. This technique can be applied to treatment techniques for patients, e.g., determination of the urgency of polypectomy with respect to a carrier and non-carrier. For healthy persons, treatment techniques can be selectively used on the basis of genetic information of each person obtained by a diagnostic test on the disease.

According to another aspect of the present invention, there is provided a transaction establishment system for analyzing whether a transaction condition including at least two transaction condition data of a first transaction party is similar to a transaction condition including at least two transaction conditions presented by any one of at least two second transaction parties to determine establishment of a transaction, thereby determining whether a transaction is established between the first transaction party and at least the two second transaction parties, comprising a first homology value calculation unit calculating a first homology value x representing a homology between the transaction condition data of the first transaction party and the transaction condition data of one of the second transaction parties, wherein the first homology value calculating unit sets n thresholds E each indicating a determination criterion for the presence/absence of a homology and calculates a first homology value xᵢ(i=1, 2, . . . , n) for each threshold Eᵢ, and a second homology value calculation unit calculating a second homology value y representing a homology between at least two transaction condition data of the first transaction party and transaction condition data of the other party who is not a target for which the first homology value calculation unit performed homology value calculation, wherein the second homology value calculating unit sets n thresholds E each indicating a determination criterion for the presence/absence of a homology and calculates a second homology value yᵢ (i=1, 2, . . . , n) for each threshold Eᵢ, wherein the establishment of the transaction is determined on the basis of the first homology value xᵢ and the second homology value yᵢ.

The present invention associated with a system (apparatus) can also be implemented as the invention of a method implemented by the apparatus.

In addition, the present invention associated with the apparatus or method can be implemented as programs for causing a computer to execute sequences corresponding to the present invention (or causing the computer to function as unit corresponding to the present invention or implement functions corresponding to the present invention), and can be implemented as a computer-readable recording medium on which the programs are recorded.

### BRIEF DESCRIPTION OF THE SEVERAL VIEWS OF THE DRAWING

FIG. 1 is a view showing the overall arrangement of a genetic analysis system according to the first embodiment of the present invention;

FIG. 2 is a view showing an example of a process of acquiring genetic data necessary for a genetic analysis according to the first embodiment;

FIG. 3 is a conceptual view showing genetic data groups stored in an amino acid sequence storage unit according to the first embodiment;

FIG. 4 is a conceptual view showing the BLAST method according to the first embodiment;

FIG. 5 is a view showing the concept of determination of the presence/absence of homologies by the BLAST method according to the first embodiment;

FIG. 6 is a view showing the relationship between the homology value hit number and the threshold E according to the first embodiment;

FIG. 7 is a view showing homology determination values Zᵢ⁽¹⁾ according to the first embodiment;

FIG. 8 is a view showing an example of a determination table used for determination result derivation in a t-test according to the first embodiment;

FIG. 9 is a view schematically showing the origin of a yeast gene speculated from the determination result obtained by a t-test according to the first embodiment;

FIG. 10 is a view showing the overall arrangement of a transaction establishment system according to the second embodiment of the present invention;

FIG. 11 is a view showing the detailed arrangement of a server according to the second embodiment;

FIGS. 12A and 12B are views showing display examples of human resource agency web pages according to the second embodiment;

FIG. 13 is a view showing the concept of determination of the presence/absence of homologies according to the second embodiment; and

FIGS. 14A and 14B are views for explaining an ordering process according to the second embodiment.

### DETAILED DESCRIPTION OF THE INVENTION

The characteristic of the present invention is that the homologies between a data group as an analysis target (analysis target data group) and other data groups are calculated, and it is determined with which data group the analysis target data group is homologous.

An embodiment of the present invention will be described below with reference to the views of the accompanying drawing.

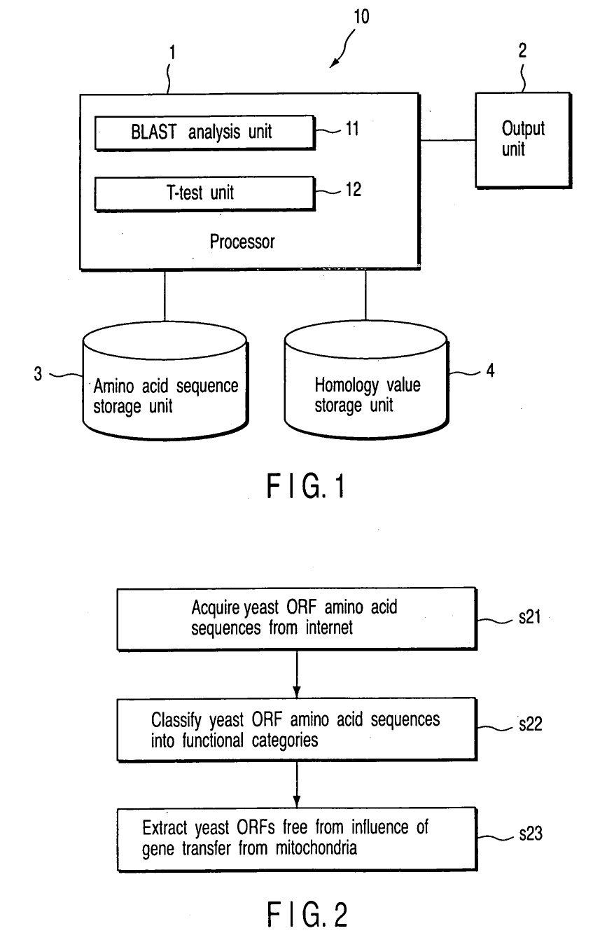

FIG. 1 is a view showing the overall arrangement of a genetic analysis system 10 according to the first embodiment of the present invention. As shown in FIG. 1, this system comprises a processor 1, an output unit 2 connected to the processor 1, an amino acid sequence storage unit 3 connected to the processor 1, and a homology value storage unit 4. The processor 1 comprises a BLAST analysis unit 11 and a t-test unit 12.

When the processor 1 reads out predetermined programs from a recording medium (not shown), the processor 1 functions as the respective units 11 and 12.

The BLAST analysis unit 11 includes a homology value calculation unit for calculating the homologies between a given data group including a plurality of data and other data groups as homology values.

The t-test unit 12 includes a homology determination value calculation unit, homology validity determination value calculation unit, and determination result derivation unit. The homology determination value calculation unit calculates homology determination values zᵢ⁽¹⁾ numerically expressing homologies, i.e., the similarities between a given data group and other data groups. The homology validity determination value calculation unit calculates homology validity determination values Z⁽²⁾ which are numerical values for determining whether or not the homology determination values Zᵢ⁽¹⁾ calculated by the homology determination value calculation unit are valid as values representing homologies. The determination result derivation unit determines which data group has data with many homologies, on the basis of the homology determination values Zᵢ⁽¹⁾ calculated by the homology determination value calculation unit and the homology validity determination values Z⁽²⁾ calculated by the homology validity value calculation unit.

A process of acquiring genetic data necessary for a genetic analysis according to this embodiment will be described next with reference to FIG. 2.

First of all, the BLAST analysis unit 11 of the genetic analysis system 10 acquires yeast ORF (Open Reading Frames) amino acid sequences through a network such as the Internet (s21). The acquired amino acid sequences are classified into 43 functional categories (s22). Yeast ORFs free from the influence of gene transfer from mitochondria are extracted from the obtained functional categories (s23). Note that 15 types of ORFs of prokaryotes excluding intracellular parasitic prokaryotes are acquired as prokaryotes.

With the above operation, the ORFs of yeasts as eukaryotes and the 15 types of ORFs of prokaryotes are obtained. The obtained ORFs are stored in the amino acid sequence storage unit 3. Note that when, for example, a yeast is an analysis target, these ORFs (genetic data) are stored such that all the ORFs of the yeast are stored as a genetic data group. For example, the prokaryotes is the analysis target, each of the ORFs (genetic data) of overall prokaryotes is stored altogether as the genetic data group. In addition, each ORF is specified by an amino acid sequence.

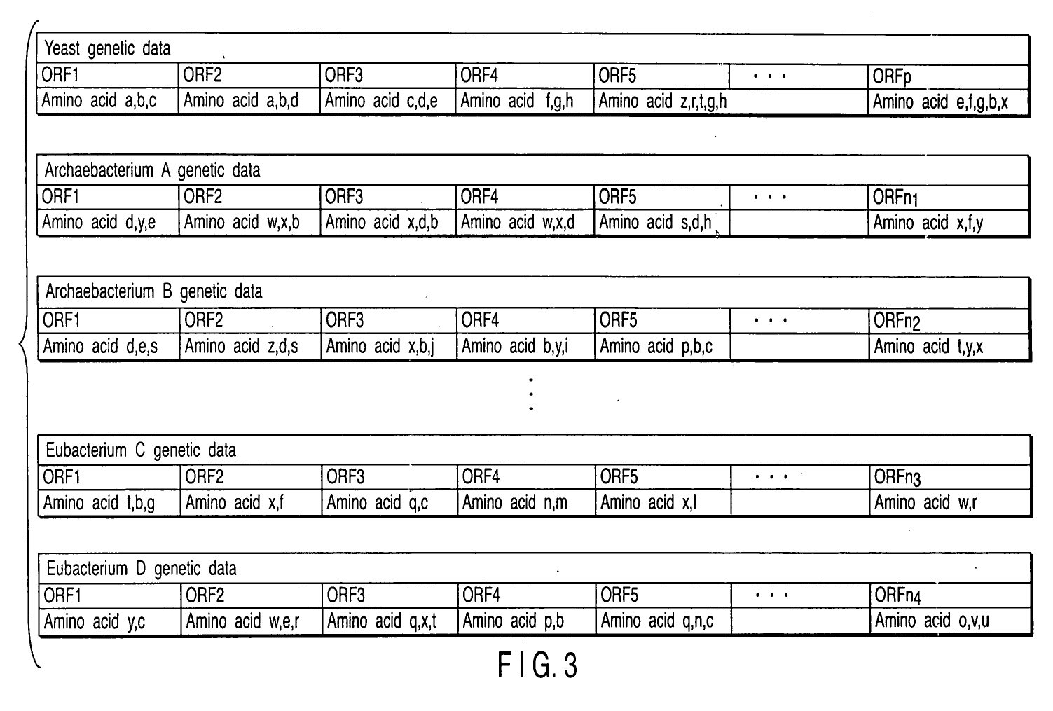

FIG. 3 is a conceptual diagram of genetic data groups stored in the amino acid sequence storage unit 3. FIG. 3 shows p yeast ORFs in total, n₁ ORFs of an archaebacterium A, n₂ ORFs of an archaebacterium B, n₃ ORFs of a eubacterium C, and n₄ ORFs of a eubacterium D.

The BLAST analysis unit 11 selects the ORFs of the yeast as an analysis target from the 16 types of ORFs obtained in the above manner. The homology value hit numbers (orthologous gene counts) between the ORFs of the yeast as the analysis target and the 15 types of ORFs are calculated by using the BLAST method.

In order to detect orthologous gene counts, first of all, E-values (negative correlations with homologies) as the indexes of homologies between all the ORFs of the yeast and all the ORFs of the respective bacteria are calculated. ORFs having the minimum E-values among the ORFs of the yeast and each bacterium are determined as orthologous genes. The number of orthologous genes (hit number) represents the homology between organisms. Such hit numbers are then calculated with respect to various kinds of thresholds (E-values).

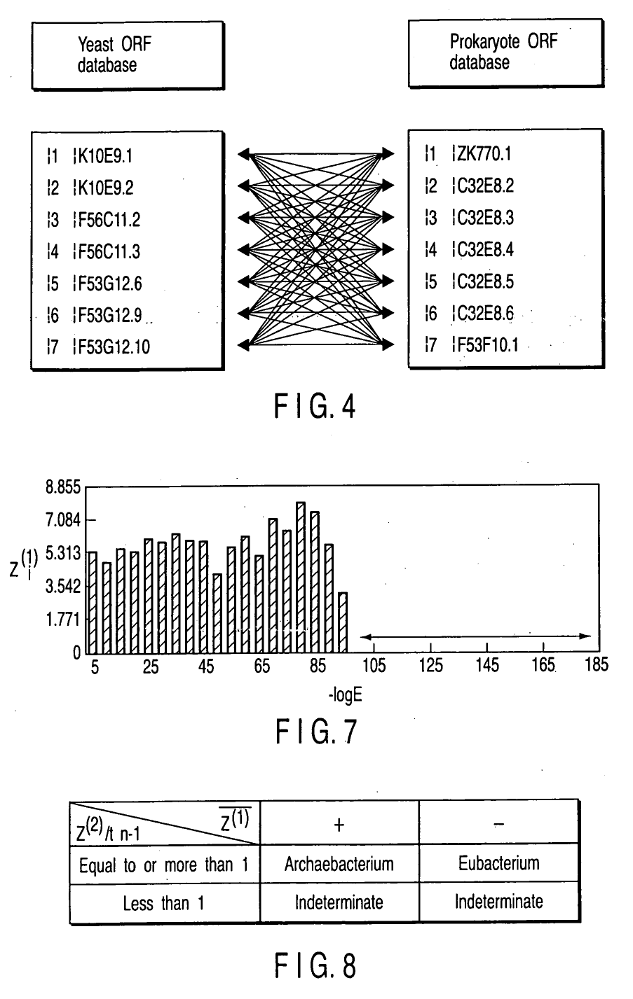

FIG. 4 is a conceptual diagram of the BLAST method. As shown in FIG. 4, homology searches are performed with respect to the yeast ORFs and the ORFs of the 15 types of prokaryotes excluding intracellular parasitic prokaryotes. In performing a homology search, first of all, the processor 1 reads out ORF 1 of the yeast and the ORFs of archaebacterium A₁ from the amino acid sequence storage unit 3. The processor 1 then calculates E-values as the indexes of homologies, and sets an ORF having the minimum E value as the first hit ORF of the archaebacterium A₁ with respect to yeast ORF 1. The processor 1 stores the corresponding ORF name and the E value at that time. This processing is repeated for all the ORFs. Remaining archaebacteria A₂, . . . , Aₙ and eubacteria are processed in the same manner as described above. The BLAST method is disclosed in, for example, “Nature Cell Biology, Vol. 3, No. 2, pp. 210-214” issued on February 1, 2001 and the entire contents of which are incorporated herein by reference. Instead of using the BLAST method as a method of comparing genes, genes can be compared at any level where a gene can be specified as information, for example, comparison can be done between amino acid sequences or base sequences or at a molecular level.

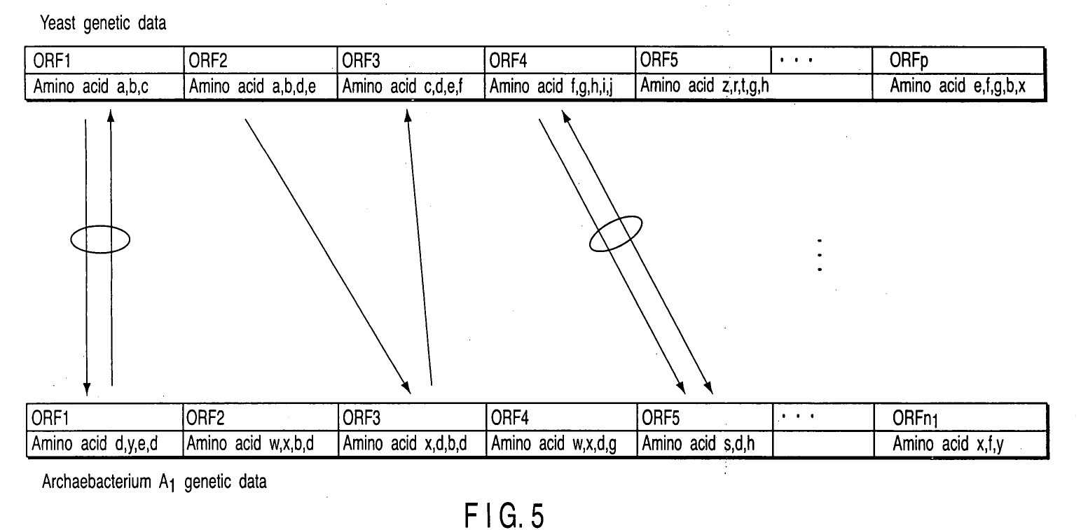

FIG. 5 is a view showing the concept of determination of the homologies between organisms which are expressed by orthologous gene counts. Referring to FIG. 5, the point of each arrow indicates the first hit gene. Note that the respective E values exceed a threshold. Each circle indicates that the respective hit genes are orthologous genes. The first hit gene for yeast ORF 2 corresponds to ORF 3 of the archaebacterium A₁. The first hit gene for ORF 3 of the archaebacterium A₁ corresponds to yeast ORF 3. Therefore, the first hit gene of yeast ORF 2 does not coincide with the first hit gene of ORF 3 of the archaebacterium A₁. The orthologous gene count represents the homology between the yeast and the archaebacterium A₁ as organisms.

Consider the yeast and archaebacterium A₁, as shown in FIG. 5. A first hit ORF-a of ORF 1 of this yeast with respect to the archaebacterium A₁ and the corresponding E-value are read out, and a first hit ORF-α of the archaebacterium A₁ with respect to the ORF-a of the yeast and the corresponding E-value are read out. When ORF 1 and the ORF-α coincide with each other and the two E-values exceed the threshold E, the corresponding genes are determined as orthologous genes, and the organism homology value hit number is incremented. Subsequently, a first hit ORF-b of ORF 2 of the yeast with respect to the archaebacterium A₁ and the corresponding E-value are read out. Similarly, the presence/absence of an orthologous gene with respect to the archaebacterium A₁ is determined. If the ORF-b corresponds to an orthologous gene, the organism homology value hit number is incremented.

In this manner, the presence/absence of orthologous ORFs is determined with respect to yeast ORF 3 to yeast ORF p. The obtained count value is stored as a homology value hit number x with respect to the archaebacterium A₁ in the homology value storage unit 4.

The above description concerns the yeast and archaebacterium A₁. The same processing is also performed for the yeast and archaebacteria A₂, . . . , Aₙ, and the resultant values are stored as homology value hit numbers x with respect to the yeast in the homology value storage unit 4.

The BLAST analysis unit 11 of the processor 1 calculates a mean value x_ of the homology value hit numbers with respect to the archaebacteria A₁, . . . , Aₙ and stores the obtained mean value in the homology value storage unit 4.

Such homology value hit numbers are also calculated with respect to other archaebacterium genetic data groups, eubacterium genetic data groups, and the like.

In detecting orthologous genes among a yeast genetic data group with p genes and genetic data groups with n_{A} archaebacteria, let xᵢⱼ be the homology value hit number obtained with respect to a given threshold Eᵢ and the jth archaebacterium.

In addition, in detecting orthologous genes among the yeast genetic data group with _ genes and genetic data groups with n_{B} eubacteria, let yᵢₖ be the homology value hit number obtained with respect to the given threshold. Eᵢ and the kth eubacterium.

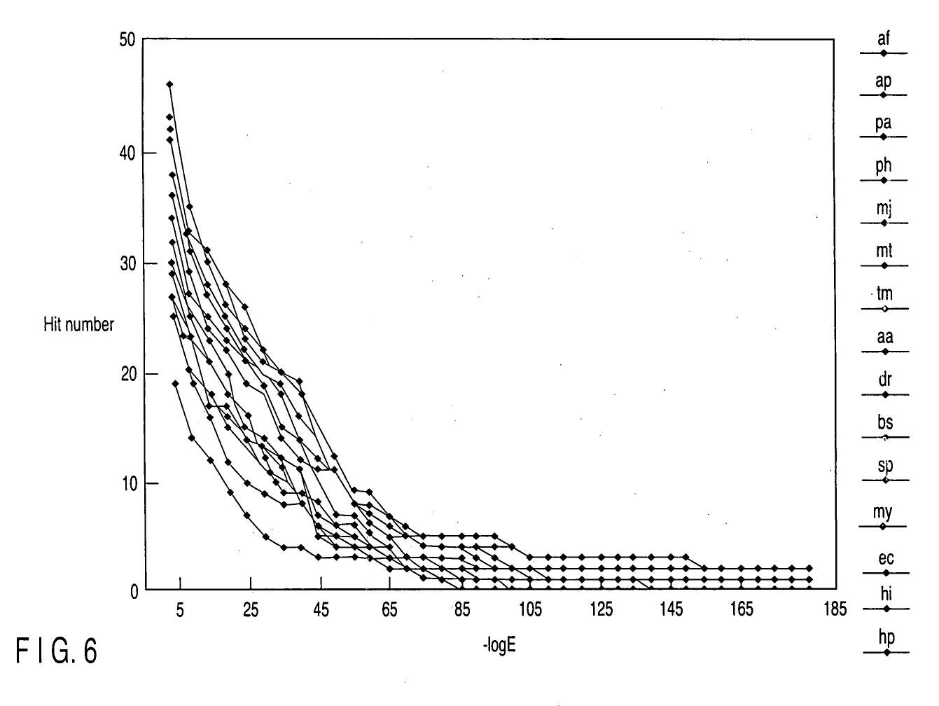

FIG. 6 shows the relationship between the homology value hit number obtained in the above manner and the threshold E. The abscissa represents −log E; and the ordinate, the homology value hit number. Referring to FIG. 6, each line graph corresponds to each bacterium to be compared with an yeast gene. Obviously, as the degree of similarity is controlled more severely, i.e., the threshold E is reduced, the number of genes that satisfy the condition, i.e., the homology value hit number, decreases.

The t-test unit 12 of the processor 1 performs the following t-test process on the basis of the various homology value hit numbers acquired in the above manner.

A T-test process comprises three processes, i.e., calculation of the homology determination values Zᵢ⁽¹⁾, calculation of the homology validity determination values Z⁽²⁾, and derivation of a determination value.

A method of calculating the homology determination values Zᵢ⁽¹⁾ will be described first by exemplifying a case wherein an analysis is made to determine with which one of an archaebacterium genetic data group and eubacterium genetic data group an yeast genetic data group shares more genes having a high homology.

Assume that with each of the above thresholds Eᵢ (i=1, 2, . . . , n), a one-sided t-test with a significant level of 5% is performed in an area where a hit number as a criteria to be satisfied is five or more.

For example, the homology determination values Zᵢ⁽¹⁾ are calculated by

$${Z}_{i}^{( 1 )} = \frac{\overline{{x}_{i}} - \overline{{y}_{i}}}{{u}_{i}} \cdot \sqrt{\frac{{n}_{A} \cdot {n}_{B}}{{n}_{A} + {n}_{B}}} ( i = 1 , 2 , … , n )$$

In the above equation, xᵢ_{_{—}} is the mean value of hit numbers xᵢⱼ (j=1, 2, . . . , n_{A}) with the ith E value, yᵢ_{₁₃} is the mean value of hit numbers Yᵢₖ (k=1, 2, . . . , n_{B}) with the ith E value, and n is the number of thresholds set for an analysis by the BLAST method. An unbiased variance uᵢ is given by

$${u}_{i} = \sqrt{\frac{1}{{n}_{A} + {n}_{B} - 2} \{ {\sum }_{j = 1}^{{n}_{A}} {( {x}_{ij} - \overline{{x}_{i}} )}^{2} + {\sum }_{k = 1}^{{n}_{B}} {( {y}_{ik} - \overline{{y}_{i}} )}^{2} \}}$$

FIG. 7 shows the result obtained by statistically processing the homology determination values Zᵢ(1) obtained in the above manner. Referring to FIG. 7, the abscissa represents −log E; and the ordinate, the homology determination value Zᵢ⁽¹⁾, which is obtained on the basis of the data shown in FIG. 6. The numbers of genes determined to have homologies are represented by a bar graph in an area where the numbers of such genes are five or more. Assume the homology determination value Zᵢ⁽¹⁾ is equal to or more than t_{nA+nB-2}(0, 10) (=1.771) or equal to or less than −t_{nA+nB-2}(0, 10) (=−1.771). In this case, the significant level is 5% (assuming that two population means are equal, the probability that the sample means differ from each other is 5% or less), and an archaebacterium or eubacterium gene group has a high homology with a budding yeast gene group with respect to each threshold E.

A method of calculating homology validity determination values Z⁽²⁾ will be described next. The homology validity determination values Z⁽²⁾ are calculated to perform the first t-test, i.e., determine whether the homology determination values Zᵢ(1) obtained upon calculating the homology determination values Zᵢ⁽¹⁾ are predominantly larger than overall Eᵢ set by t_{nA+nB-2}(0, 10) (=1.771), smaller than −t_{nA+nB-2}(0, 10) (=−1.771), or neither. For this purpose, the homology validity determination values Z⁽²⁾ and tₙ₋₁(0, 10) are calculated with a degree n−1 of freedom by

$${Z}^{( 2 )} = \frac{\vert \overline{{Z}^{( 1 )}} \vert - {t}_{{n}_{A} + {n}_{B - 2}} ( 0.10 )}{s / \sqrt{( n - 1 )}}$$

where s is the standard deviation of the homology determination values Zᵢ⁽¹⁾, and Zᵢ(1)_ is the mean value of Zᵢ(1). If the homology validity determination value Z⁽²⁾ is larger than a reference value tₙ₋₁, it can be determined that Zᵢ⁽¹⁾ is larger than t_{nA+nB-2} with a significant level of 5%.

Determination result derivation is performed on the basis of the homology determination values Zᵢ(1) and homology validity determination values Z⁽²⁾ calculated in the above manner. The determination result is derived by using the determination table shown in FIG. 8. As shown in FIG. 8, if Z⁽²⁾/tₙ₋₁(0, 10) is equal to or more than 1 and the mean value of Zᵢ⁽¹⁾ is positive, it can be determined that the yeast ORF group in the corresponding category includes more ORFs having homologies with the archaebacterium than with the eubacterium. If Z⁽²⁾/tₙ₋₁(0, 10) is equal to or more than 1 and the mean value of Zᵢ⁽¹⁾ is negative, it can be determined that the yeast ORF group in the corresponding category includes more ORFs having homologies with the eubacterium than with the archaebacterium. If Z⁽²⁾/tₙ₋₁(0, 10) is smaller than 1, it is determined that whether the yeast ORF group has homologies with any specific bacterium cannot be determined.

According to this arrangement, the homologies of many gene groups can be evaluated altogether. Since there is no need to select any representative genes to draw a phylogenetic tree as in the prior art, the characteristics of gene groups can be understood more accurately.

For example, the origin of a eukaryote can be explored by using this method. Conventionally, the origin has been estimated by forming a phylogenetic tree mainly concerning rRNA and a small number of proteins. According to this estimation, it has been expected that gene groups based on DNA replication, transfer, translation, and the like originating from archaebacteria, gene groups based on energy metabolism originating from the symbiosis of mitochondria, and other gene groups have a mosaic structure of archaebacteria and eubacteria. There have been also other hypotheses.

In order to examine the origins of the respective functions of an yeast, the ORFs (Open Reading Frames) of 15 types of bacteria excluding intracellular parasitic bacteria are compared with the yeast ORFs classified into functional categories. That is, an yeast genetic data group is set as a target for the above BLAST analysis and t-test analysis, and an archaebacterium genetic data group and eubacterium genetic data group are set as comparison targets. As a result, it was found that 20 of the yeast gene groups classified into 43 functional categories include many genes having high homologies with one of an archaebacterium and a eubacterium. Since it is generally known that evolutionarily close organisms hold many genes having high homologies, the respective origins can be estimated for the respective functional categories of the yeast gene.

More specifically, this BLAST analysis and t-test revealed that gene groups based on DNA synthesis/replication, transfer, translation, postmeiotic fusion, cell cycle adjustment, endoplasmic reticulum formation, nucleation, and the like shared many genes having high homologies with archaebacterium ORFs, whereas gene groups based on energy metabolism, various kinds of metabolisms, material transportation into cells, stress response, detoxication, and ion homeostasis shared many genes having high homologies with eubacterium ORFs.

It is therefore thought that genes associated with nuclei (genetic information) originating from archaebacterium genes, and genes associated with cytoplasm (homeostasis) originating from eubacterium genes. This means that one of the gene groups was replaced with the other organism.

These results indicate that the nucleus of a eukaryote originating from the symbiosis of an archaebacterium with a eubacterium. That is, when the relationship between gene groups is estimated by homology determination, it is highly possible that a eukaryote originates from the symbiosis of an archaebacterium with a eubacterium.

This theory can be thought as follows along with each widely accepted theory.

First of all, with regard to the theory that a gene of eubacterium origin was created as a result of the symbiosis of mitochondria, since genes associated with mitochondria are deleted from analysis data, it can be concluded that the symbiosis of mitochondria has no influence.

With regard to the theory that such a gene was created due to the accumulation of gene horizontal transfer, since gene horizontal transfer is caused by a chance factor, it can be concluded-that it is quite unlikely that gene horizontal transfer occurs for only a specific function, and an entire gene group of the function is replaced with another gene group.

With regard to the theory that such a gene was created as a result of the invasion and symbiosis of an archaebacterium in and with a eubacterium, the replacement of a gene group associated with a specific function indicates that only the gene group was in a special environment in which it was easily replaced. A nucleation process based on intracellular symbiosis satisfies this condition, and it is widely recognized that evolutionary formation of mitochondria and chloroplast originates from intercellular symbiosis. It can therefore be concluded that this theory is plausible. It can therefore be thought that a eukaryote was created as a result of the invasion and symbiosis of an archaebacterium in and with a eubacterium.

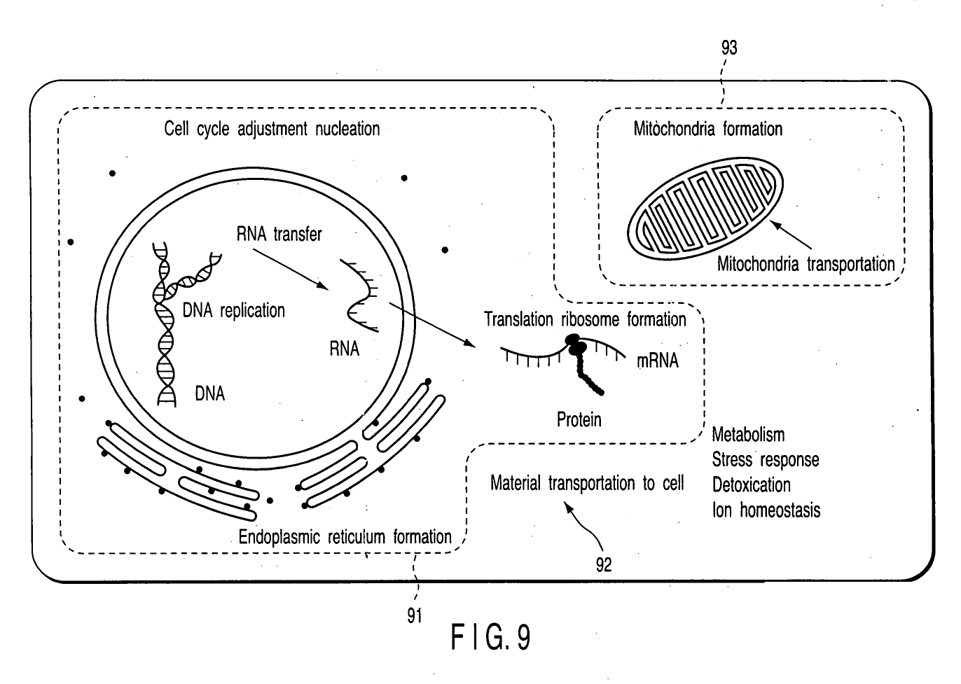

More specifically, the following estimation can be made. As an archaebacterium which has undergone an intracellular symbiosis with a eubacterium kept using the metabolic product synthesized by the eubacterium, the archaebacterium lost a gene group necessary for the synthesis. The cell cycle of the eubacterium is dominated by the archaebacterium. Finally, even the process from gene replication to protein synthesis depended on the archaebacterium, and the remaining genes gradually shifted. FIG. 9 schematically shows this. As shown in FIG. 9, it is thought that a nucleus-associated gene 91 associated with cell cycle adjustment, nucleation, DNA replication, RNA transfer, endoplasmic reticulum formation, translation, ribosome formation, and the like originates from a archaebacterium gene, and a cytoplasm 92 associated with material transportation to a cell, metabolism, stress response, detoxication, ion homeostasis, and the like is associated with a gene group originating from a eubacterium gene. In addition, it is thought that mitochondria 93 associated with mitochondria formation, mitochondria transportation, and the like originates from the symbiosis of mitochondria.

In this manner, a gene group is segmented up to the domain level to improve the detectivity of homology analysis. In addition, an ancestral gene is reproduced as a protein, and its activity is checked, thereby greatly improving the reliability of estimation concerning the origin of the gene.

As described above, according to this embodiment, the evolutionary relationship between three organisms, i.e., a eukaryote, eubacterium, and archaebacterium, can be clarified by using yeast and bacterium ORF data whose entire genome sequences are known. In addition, the homologies between all organisms can be stably and easily determined independently of set conditions for thresholds by analyzing the homologies using the ORF data of a human and other organisms.

The present invention is not limited to the above embodiment. Although homology value hit numbers are calculated by using the BLAST method, it is obvious that other homology value calculation methods can also be used. In a t-test, the significant level is set to 5%. However, the present invention is not limited to this, and a test may be conducted on the basis of a different significant level. In addition, a test may be conducted by using a testing technique other than t-test.

In addition, for example, the present invention can be applied to comparing an enormous number of gene groups in accordance with a given purpose, selecting a specific gene, or specifying a selected gene. When there are a plurality of gene groups as targets, calculation with the respective thresholds (E values) makes it easy to compare the homologies/similarities between a gene group having a certain characteristic and the genes of each of a plurality of groups having an enormous number of genes and to extract and specify target genes. Furthermore, this technique can specify a gene having a unique characteristic among a target gene group. For example, the technique can be applied to extraction of genes introduced by so-called horizontal transfer instead of transfer from parents to children in the evolutionary process of an organism.

A specified gene can be extracted from an analyzed organism, and its DNA sequence can be known. Alternatively, similar genes can be searched out, and the functions of the genes can be estimated on the basis of information about the similar genes. The above technique can also be applied to selection of human resources according to a given purpose. That is, data which satisfies a given condition can be selected/specified from among a group (data group) including an enormous number of data. Alternatively, data can be selected/specified while the degree of the condition is changed. By analyzing the homologies between groups, a pair of data which satisfy mutual conditions can also be selected.

Currently, after the elucidation of the human genome, there has been a big trend toward placing great importance on selecting/specifying genes that cause diseases. A gene candidate is produced as a DNA fragment from an actual human DNA sample by the PCR gene amplification method, and a protein synthesized by the gene is actually produced by an expression system such as Escherichia coli. The activity of the protein is then observed or an antibody for the protein is produced. The antibody can be effectively used for a disease detection method or the like by being actually applied to a pathological section of the human or the like. In order to specify a gene that causes a disease, a morbid area of the tissue of the patient is specified or the genes of a healthy person are analyzed. This can discriminate a person who is vulnerable to the disease. This technique can be applied to treatment techniques for patients, e.g., determination of the urgency of polypectomy with respect to a carrier and non-carrier. For healthy persons, treatment techniques can be selectively used on the basis of genetic information of each person obtained by a diagnostic test on the disease.

If data other than genetic data are used as data groups as homology comparison targets, the present invention can also be applied to analysis/selection of human resource data, similarity determination of merchandise, meteorological information analysis, and the like. That is, the present invention can be applied to anything as long as the degrees of similarities between data groups each comprising a plurality of data are analyzed.

(Second Embodiment)

This embodiment is a modification of the first embodiment. In this embodiment, the homology analysis of data in the first embodiment is applied to intermediacy services for transaction establishment. The embodiment will be described by taking human resource agency as an example.

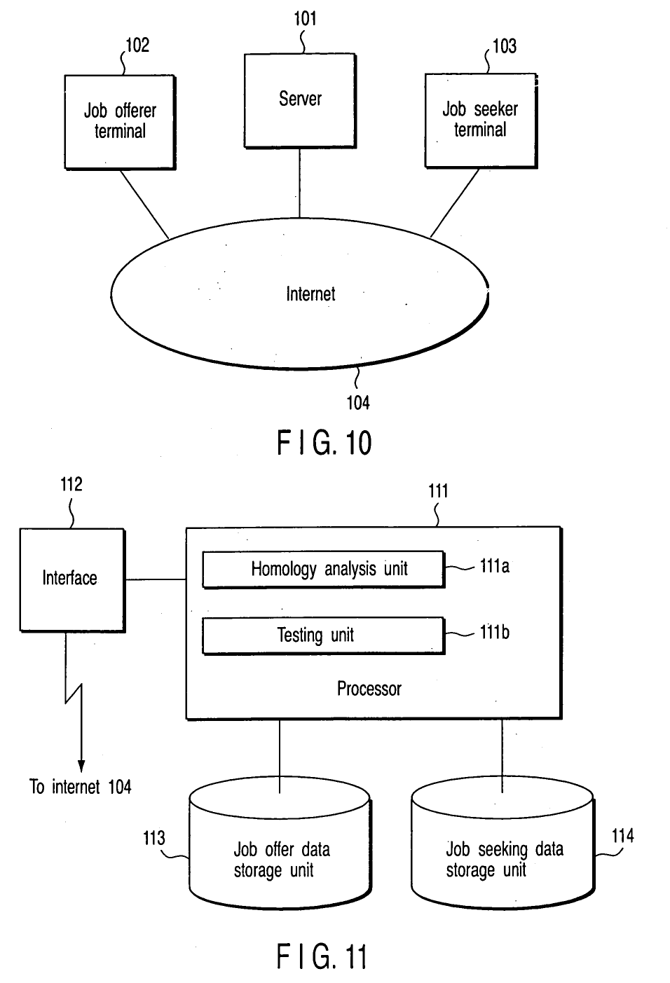

FIG. 10 is a view showing the overall arrangement of a transaction establishment system according to this embodiment. As shown in FIG. 10, a server 101, job offerer terminal 102, and job seeker terminal 103 are connected to the Internet 104. The server 101 is managed by a personnel agency who performs human resource agency according to this embodiment. The job offerer terminal 102 is used by a job offerer who wants to acquire a desired person through this human resource agency service. The job seeker terminal 103 is used by a job seeker who wants to find employment in a desired company through the human resource agency service.

The Internet 104 may be replaced with any network designed to exchange information by transmission/reception of data. Any connection form can be used regardless of whether it is wired or wireless.

FIG. 11 is a view showing the detailed arrangement of a server 1. As shown in FIG. 11, the server 1 comprises a processor 111, an interface 112 which is connected to the processor 111 and controls transmission/reception of information to/from a network 4, a job offer data storage unit 113 which stores job offer data obtained from job offerers, and a job seeking data storage unit 114 which stores job seeker data obtained from job seekers. The processor 111 has a homology analysis unit 111a and testing unit 111b.

The homology analysis unit 111a has a homology value calculation unit for calculating the homologies between a given data group including a plurality of data and other data groups as homology values.

The testing unit 111b has the same arrangement as that of the t-test unit 12 in the first embodiment, and functions as a homology determination value calculation unit, homology validity determination value calculation unit, and determination result derivation unit.

A human resource agency method according to this embodiment will be described next.

First of all, the server 1 is set to allow job offerers and job seekers to browse human resource agency web pages through the Internet 104. More specifically, human resource agency web page files are stored in a storage unit (not shown) in the server 1 so as to allow data acquisition through the Internet 104. Note that the human resource agency web page files include a job offerer web page file and job seeker web page file. In response to a connection request from a job offerer, the job offerer web page file is transmitted. In response to a connection request from a job seeker, the job seeker web page file is transmitted.

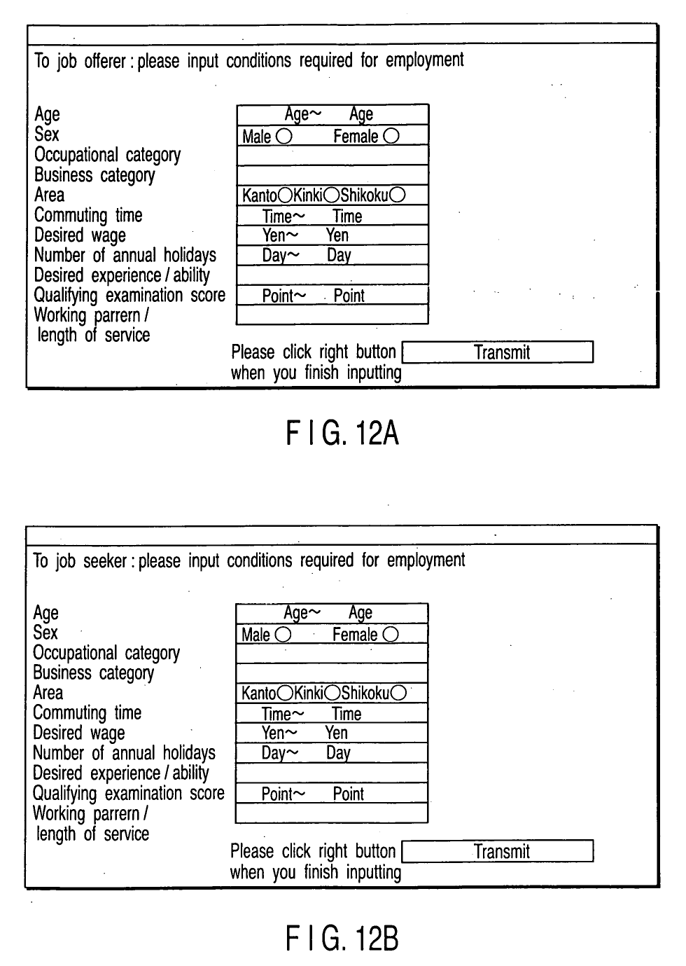

When the job offerer issues a connection request for the human resource agency web page in the server 1 from the job offerer terminal 102, the server 1 transmits the human resource agency web page file to the job offerer terminal 102. For example, a job offerer web page like the one shown in FIG. 12A is displayed on the display unit (not shown) of the job offerer terminal 102. The job offerer inputs job offer data by using the job offerer terminal 102 in accordance with the window shown in FIG. 12A. The following are input examples of the job offer data: age, sex, occupational category, business category, area, commuting time, desired wage, number of annual holidays, desired experience and ability, e.g., qualifying examination score, working pattern, and length of service. The input job offer data is transmitted to the job offerer terminal 102. The server 101 registers the received job offer data in the job offer data storage unit 113. Note that the respective data items included in job offer data are preferably converted into numerical data when registered in the job offer data storage unit 113. Each data item may be set to a unique value, e.g., 250,000 yen in monthly income, or may be set to a value in a predetermined range, e.g., 250,000 yen to 300,000 yen.

When the job seeker issues a connection request for the human resource agency web page in the server 1 from the job seeker terminal 103, the server 1 transmits the human resource agency web page file to the job seeker terminal 103. For example, a job seeker web page like the one shown in FIG. 12B is displayed on the display unit of the job seeker terminal 103. The job seeker inputs job seeking data by using the job seeker terminal 103 in accordance with the window shown in FIG. 12B. The respective data included in the job seeking data are common to those of the job offer data. The input job seeking data is transmitted to the job seeker terminal 103. The server 101 stores the received job seeking data in the job seeking data storage unit 114. Note that the respective data items included in job seeking data are preferably converted into numerical data when registered in the job seeking data storage unit 114. Each data item may be set to a unique value, e.g., 250,000 yen in monthly income, or may be set to a value in a predetermined range, e.g., 250,000 yen to 300,000 yen.

The server 1 registers a plurality of job offer data and a plurality of job seeking data in the job offer data storage unit 113 and job seeking data storage unit 114, respectively, through the above process. After this registration, the homology analysis unit 111a of the server 1 selects the job offer data of a given job offerer as an analysis target. The homology analysis unit 111a then calculates the homology value hit numbers between the job offer data and the respective job seeking data. This homology value hit number calculation is the same processing as that performed by the BLAST analysis unit 11 in the first embodiment. When the yeast ORFs in the first embodiment are replaced with the respective data items included in the job offer data of the given job offerer in the second embodiment, the homology value hit numbers between the job offer data and the respective job seeking data are calculated.

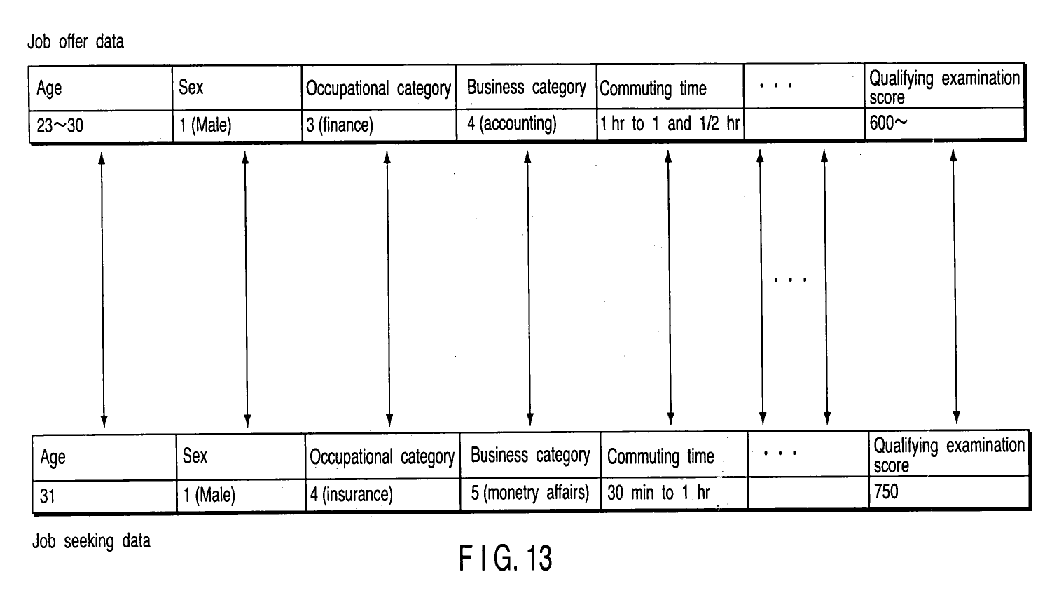

In the case of human resource agency in this embodiment, the respective data items are not pieces of fragmentary information which are not orderly arranged like ORFs in the first embodiment. Therefore, it is only required to determine whether or not there are hits between the same data items, e.g., monthly incomes and commuting times. FIG. 13 is a view showing the concept of determination of the presence/absence of homologies. As shown in FIG. 13, there is no need to determine hit numbers between different types of data items unlike in FIG. 5.

Such calculation of homology value hit numbers is performed by using a plurality of thresholds for all the job seeking data as in the first embodiment, and the obtained homology value hit numbers are stored in the job offer data storage unit 113 in association with the job offer data as the analysis target.

The relationship between the homology value hit numbers and the thresholds obtained in the above manner is similar to that shown in FIG. 6 in the first embodiment. The relationship between a given job offerer and each job seeker is represented by a line graph. The abscissa represents the severity of the condition of each data item of the job offer data, and the ordinate represents which job seeker satisfies which conditions. The data representing the relationship between the homology value hit numbers and the thresholds is transmitted to the job offerer terminal 102 as the analysis target to be displayed on the display unit of the job offerer terminal 102. This allows the job offerer to select a required person.

The above description has exemplified the case wherein once job offer data is registered, homology determination is performed without changing the conditions. However, if the condition of a data item, e.g., monthly income, is changed, the hit number of each job seeker varies. With this variation in hit number, the line graph corresponding to each job seeker varies. As described above, this makes it easy to select candidates who vary when the condition of a data item is changed.

The above homology determination for the job offer data of a given job offerer can be performed with respect to the job seeking data of a given job seeker. This makes it possible for the job offerer to select a job seeker which satisfies the conditions. In this case as well, a curve representing the relationship between the homology value hit numbers and the thresholds, like the one shown in FIG. 6, can be obtained. Such data representing the relationship between the homology value hit numbers and the thresholds is transmitted to the job seeker terminal 103 as an analysis target to be displayed on the display unit of the job seeker terminal 103. This allows the job offerer to select a required person.

In the above case, a person is selected on the basis of the relationship between the homology value hit numbers and the thresholds. In many cases, however, it is difficult to select a person on the basis of only the relationship curves shown in FIG. 6. For this reason, the testing unit 111b of the processor 111 executes a test process like a t-test process in the first embodiment. The test process comprises an ordering process in addition to three processes, i.e., calculation of homology determination values, calculation of homology validity determination values, and determination result derivation like those performed by the t-test unit 12 in the first embodiment. Calculation of homology determination values, calculation of homology validity determination values, and determination result derivation are the same as those in the first embodiment, and hence a detailed description thereof will be omitted.

In a determination result derivation process, it can be determined which data group has homology. This makes it possible to select persons who satisfy given conditions or severer conditions.

Assume that a job offerer can know which person satisfies conditions most and a job seeker can know which person satisfies conditions most and can also know a specific order in which persons satisfy the conditions presented by himself/herself. In this case, employment and job search activities can be effectively done.

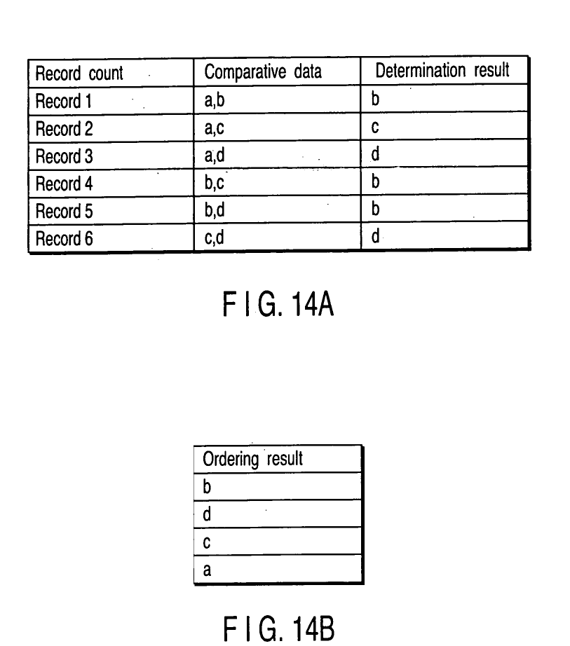

For this reason, an ordering process with respect to conditions for persons is executed on the basis of the data obtained by a determination result derivation process. When, for example, an ordering process is performed on the basis of the determination results on the respective job seekers for a given job offerer, determination results like those shown in FIG. 14A can be obtained by a determination result derivation process. The respective job seekers are arranged in decreasing order of homology on the basis of the determination results. Ordering can be easily done by, for example, assuming that a given job seeker is at a given ordinal level, and combining the determination results with the assumption. Assume that a job seeker a is in the first place in the case shown in FIG. 14A. In this case, according to the information of record 1, a job seeker b ranks higher than the job seeker a, the job seeker b is set in the first place, and the job seeker a is set in the second place. According to the information of record 2, since a job seeker c ranks higher than the job seeker a, the job seekers c and b are set in the first or second place, and the job seeker a is set in the third place. In this manner, ordering can be done on the basis of the respective determination results. FIG. 14B shows the obtained ordering process result.

Obviously, this ordering process can be applied to ordering of job offerers based on the job seeking data of a given job seeker as well as ordering of job seekers based on the job offer data of a given job offerer.

Transmitting such an ordering result to the job offerer terminal 102 or job seeker terminal 103, together with data representing the relationship curves between the homology value hit numbers and the thresholds, will further facilitate selection of persons. Obviously, when the condition of each data item of job offer data or job seeking data is changed, ordering can be done in accordance with a change in condition. This makes it possible to grasp how the conditions presented by persons are changed when conditions are changed, thereby further facilitating selection of persons.

In this embodiment, data are acquired by using the job offerer terminal 102 and job seeker terminal 103 through the Internet 104, and human resource agency processing is performed on the basis of the data. However, the present invention is not limited to this. Human resource agency processing similar to that described above can be performed by receiving information from job offerers and job seekers through telephones, FAXs, paper media, and the like and inputting the information to the server 1.

In this embodiment, the transaction establishment system has been described with its object being limited to human resource agency. However, the embodiment can be easily applied to systems designed to handle transactions of merchandise and services other than human resources by replacing job offer data and job seeking data necessary for human resource agency with other data.

When, for example, this embodiment is applied to a merchandise transaction system, the above job offer data and job seeking data may be replaced with conditions for purchasing or selling merchandise (e.g., types of merchandise, functions of merchandise, prices of merchandise, periods of time required to obtain merchandise, and the like). When matrimonial services are to be provided, the above job offer data and job seeking data may be replaced with various conditions required for male and female marriage partners (e.g., age, yearly income, height, weight, personality, address, family make-up, and the like).

In application of this embodiment to any transaction system, with regard to information which is difficult to digitize, the precision of homology determination can be improved by assigning close numerical values to data close in terms of concept or condition. For example, occupational categories in human resource agency are difficult to digitize. Consider, for example, the manufacturing industry, financial industry, and insurance industry as occupational categories. In this case, the financial and insurance industries are relatively close concepts, and many job seekers think that they may arbitrarily select either of them. In contrast to this, the manufacturing industry is a concept which is not relatively close to the financial and insurance industries. In such a case, the numerical values “1” and “2” may be assigned to the financial and insurance industries, and the numerical value “20” greatly different from “1” and “2” may be assigned to the manufacturing industry. This makes it possible to determine homology in accordance with the magnitudes of numerical values. The precision of homology determination can also be improved by methods other than this method of assigning numerical values, e.g., a data selection method of making job seekers or job offerers select a plurality of data (e.g., both the financial and insurance industries) and calculating hit numbers depending on whether or not selected data coincide with each other.

In addition to transaction establishment, this embodiment can also be applied to any systems designed to perform matching between wishes mutually presented from two different groups, e.g., matching between male and female members who wish to marry. Obviously, since one group may be a single group, this embodiment can also be applied to an auction or the like.

The present invention can also be applied to other techniques.

For example, the present invention can be applied to a technique of acquiring image data such as satellite photographs at a plurality of times, and determining the homologies between them in the same manner as in the above embodiment, thereby easily specifying a portion that has undergone a change.

In addition, if, for example, fires are imaged and compared with each other, fire detection can be performed. That is, the present invention can be applied to fire prevention. Furthermore, pieces of information of airplanes which momentarily change on a radar in a control tower for aircraft may be compared with each other to instantaneously discriminate a new change or the like. The present invention can also be applied to a technique of instantaneously detecting any change at a point upon launching of a missile or the like.

If a weather map is acquired as an image, a weather forecast can be made by comparing the map with past-weather maps.

In addition, a more accurate weather forecast can be made by acquiring not only a weather map but also temperatures, barometric pressures, weather conditions, and the like at the respective points captured as the image and performing homology determination by comparing the acquired data with past data.

Furthermore, images of many fingerprints, likenesses, and the like are decomposed into characteristic portions and the like. The configurations are then converted into patterns or most similar portions are discriminated and specified. In this case, discriminated cases are extracted while the tolerances for the respective elements are changed. This allows the present invention to be applied to specifying criminals, anthropological comparison, and the like.

Image data may be divided into digital signals, and homology determination may be performed between the divided digital signals. Alternatively, signals subjected to this homology determination may be replaced with other signals, and the two signals are compared with each other to easily specify portions corresponding to different signals. If, for example, audio signals and the like are divided by a predetermined frequency and the resultant signals are compared with each other, musical similarity can be determined. In addition, if, for example, pieces of character information of novels or the like are divided by a predetermined length and the resultant pieces of information are compared with each other, the similarity between expressions and words in use in the novels can be determined. This allows the present invention to be applied to determination of the similarity between two creative works, plagiarism, or the like. Obviously, the present invention can be applied to not only determination of such creativity but also the linguistic field, in which the differences in language and the like between children and adults or between different areas are compared, and features are extracted, thereby extracting common and different points as concrete elements.

Furthermore, the present invention can be applied to lesion detection/determination using roentgenograms, MRI (Magnetic Resonance Imaging), echograms, and the like.

As has been described in detail above, according to the present invention, many data can be compared with each other quantitatively and accurately.

As has been described above, the present invention can be effectively applied to the field of homology analysis systems, the field of homology analysis methods, the field of homology analysis programs, and the field of transaction establishment systems which analyze the homologies between data groups each including a plurality of data.

What is claimed is:

1. A homology analysis system for analyzing whether an analysis target data group is similar to a first data group or a second data group wherein the first and second data groups is different from the analysis target data group, comprising:
a first homology value calculation unit calculating a first homology value x representing a homology between data included in the analysis target data group and the first data group respectively, wherein the first homology value calculating unit sets n thresholds E each indicating a determination criterion for the presence/absence of a homology and calculates a first homology value xᵢ (i=1, 2, . . . , n) for each threshold Eᵢ;
a second homology value calculation unit calculating a second homology value y representing a homology between data included in the analysis target data group and the second data group respectively, wherein the second homology value calculating unit sets n thresholds E each indicating a determination criterion for the presence/absence of a homology and calculates a second homology value yᵢ (i=1, 2, . . . , n) for each threshold Eᵢ; and
homology determination unit determining to which one of the first and second data groups the analysis target data group is similar on the basis of a relationship between the first homology value xᵢ, the second homology value yᵢ, and the number n of thresholds.

2. A homology analysis system according to claim 1, wherein
the first homology value calculation unit determines the presence of a homology if a homology between data included in the analysis target data group and the first data group, respectively, is not less than the threshold E, and calculates the number of data having homologies as the first homology value x, and
the second homology value calculation unit determines the presence of a homology if a homology between data included in the analysis target data group and the second data group, respectively, is not less than the threshold E, and calculates the number of data having homologies as the second homology value y.

3. A homology analysis system according to claim 1, wherein
when the first data group has n_{A} data, and the second data group has n_{B} data,
the first homology value calculation unit calculates a first homology value xᵢⱼ (j=1, 2, n_{A}) for each data of the first data group with respect to one threshold Eᵢ, and calculates a mean value xᵢ_{_{—}} of the calculated first homology values xᵢ with respect to the threshold Eᵢ,
the second homology value calculation unit calculates a second homology value yᵢⱼ (j=1, 2, n_{B}) for each data of the second data group with respect to one threshold Eᵢ, and calculates a mean value yᵢ_{_{—}} of the calculated second homology values yᵢ with respect to the threshold Eᵢ, and
the homology determination unit calculates a homology determination value Zᵢ⁽¹⁾ indicating similarity to one of the first data group and the second data group according to
$${Z}_{i}^{( 1 )} = \frac{\overline{{x}_{i}} - \overline{{y}_{i}}}{{u}_{i}} \cdot \sqrt{\frac{{n}_{A} \cdot {n}_{B}}{{n}_{A} + {n}_{B}}} ( i = 1 , 2 , … , n )$$
$$when {u}_{i} = \sqrt{\frac{1}{{n}_{A} + {n}_{B} - 2} \{ {\sum }_{j = 1}^{{n}_{A}} {( {x}_{ij} - \overline{{x}_{i}} )}^{2} + {\sum }_{k = 1}^{{n}_{B}} {( {y}_{ik} - \overline{{y}_{i}} )}^{2} \}}$$

4. A homology analysis system according to claim 1, wherein
when the first data group has n_{A} data, and the second data group has n_{B} data,
the first homology value calculation unit calculates a first homology value xᵢⱼ (j=1, 2, n_{A}) for each data of the first data group with respect to one threshold Eᵢ, and calculates a mean value xᵢ_{_{—}} of the calculated first homology values xᵢ with respect to the threshold Eᵢ,
the second homology value calculation unit calculates a second homology value yᵢⱼ (j=1, 2, n_{B}) for each data of the second data group with respect to one threshold Eᵢ, and calculates a mean value yᵢ_{_{—}} of the calculated second homology values yᵢ with respect to the threshold Eᵢ,
the homology determination unit calculates a homology determination value Zᵢ⁽¹⁾ indicating similarity to one of the first data group and the second data group according to
$${Z}_{i}^{( 1 )} = \frac{\overline{{x}_{i}} - \overline{{y}_{i}}}{{u}_{i}} \cdot \sqrt{\frac{{n}_{A} \cdot {n}_{B}}{{n}_{A} + {n}_{B}}} ( i = 1 , 2 , … , n )$$
$$when {u}_{i} = \sqrt{\frac{1}{{n}_{A} + {n}_{B} - 2} \{ {\sum }_{j = 1}^{{n}_{A}} {( {x}_{ij} - \overline{{x}_{i}} )}^{2} + {\sum }_{k = 1}^{{n}_{B}} {( {y}_{ik} - \overline{{y}_{i}} )}^{2} \}}$$
and
the homology analysis system further comprises determination result derivation unit determining that the analysis target data group has many data having homologies with the first data group, if the homology determination value Zᵢ⁽¹⁾ is larger than t_{α}(0, 10) wherein the homology determination value Zᵢ⁽¹⁾ is in accordance with a t-distribution and ax is a degree of freedom.

5. A homology analysis system according to claim 1, in which
when the first data group has n_{A} data, and the second data group has n_{B} data,
the first homology value calculation unit calculates a first homology value xᵢⱼ (j=1, 2, n_{A}) for each data of the first data group with respect to one threshold Eᵢ, and calculates a mean value xᵢ_{_{—}} of the calculated first homology values xᵢ with respect to the threshold Eᵢ,
the second homology value calculation unit calculates a second homology value yᵢⱼ (i=1, 2, . . . , n_{B}) for each data of the second data group with respect to one threshold Eᵢ, and calculates a mean value yᵢ_{_{—}} of the calculated second homology values yᵢ with respect to the threshold Eᵢ,
the homology determination unit calculates a homology determination value Zᵢ⁽¹⁾ indicating similarity to one of the first data group and the second data group according to
$${Z}_{i}^{( 1 )} = \frac{\overline{{x}_{i}} - \overline{{y}_{i}}}{{u}_{i}} \cdot \sqrt{\frac{{n}_{A} \cdot {n}_{B}}{{n}_{A} + {n}_{B}}} ( i = 1 , 2 , … , n )$$
$$when {u}_{i} = \sqrt{\frac{1}{{n}_{A} + {n}_{B} - 2} \{ {\sum }_{j = 1}^{{n}_{A}} {( {x}_{ij} - \overline{{x}_{i}} )}^{2} + {\sum }_{k = 1}^{{n}_{B}} {( {y}_{ik} - \overline{{y}_{i}} )}^{2} \}}$$
and
the homology analysis system further comprises determination result derivation unit determining that the analysis target data group has many data having homologies with the second data group, if the homology determination value Zᵢ⁽¹⁾ is smaller than −t_{α}(0, 10) wherein the homology determination value Zᵢ⁽¹⁾ is in accordance with a t-distribution and α is a degree of freedom.

6. A homology analysis system according to claim 4, wherein the determination result derivation unit further comprises homology validity determination unit calculating a homology validity determination value Z⁽²⁾ given by
$${Z}^{( 2 )} = \frac{\vert \overline{{Z}^{( 1 )}} \vert - {t}_{{n}_{A} + {n}_{B - 2}} ( 0.10 )}{s / \sqrt{( n - 1 )}}$$
where s is a standard deviation of Zᵢ⁽¹⁾ and Zᵢ⁽¹⁾_ is a mean value of Zᵢ⁽¹⁾, and determining that the homology determination value Zᵢ⁽¹⁾ is an invalid value, if the homology validity determination value Z⁽²⁾ is less than a predetermined value tₙ₋₁(0, 10).

7. A homology analysis system according to claim 5, wherein the determination result derivation unit further comprises homology validity determination unit calculating a homology validity determination value Z⁽²⁾ given by
$${Z}^{( 2 )} = \frac{\vert \overline{{Z}^{( 1 )}} \vert - {t}_{{n}_{A} + {n}_{B - 2}} ( 0.10 )}{s / \sqrt{( n - 1 )}}$$
where s is a standard deviation of Zᵢ⁽¹⁾ and Zᵢ⁽¹⁾_ is a mean value of Zᵢ⁽¹⁾, and determining that the homology determination value Zᵢ⁽¹⁾ is an invalid value, if the homology validity determination value Z⁽²⁾ is less than a predetermined value tₙ₋₁(0, 10).

8. A homology analysis system according to claim 6, wherein the degree of freedom α is n_{A}+n_{B}−2.

9. A homology analysis system according to claim 7, wherein the degree of freedom α is n_{A}+n_{B}−2.

10. A homology analysis system according to claim 1, wherein the first and second homology value calculation unit calculates the homology values xᵢ and yᵢ by a BLAST method.

11. A homology analysis system according to claim 1, wherein the analysis target data group, the first data group, and the second data group are data representing gene sequences.

12. A homology analysis method of analyzing whether an analysis target data group is similar to a first data group or a second data group wherein the first and second data groups is different from the analysis target data group, comprising:
calculating a first homology value x representing a homology between data included in the analysis target data group and the first data group, respectively, wherein the calculating the first homology value x includes setting n thresholds E each indicating a determination criterion for the presence/absence of a homology and calculating the first homology value x as a first homology value xᵢ (i=1, 2, . . . , n) for each threshold Eᵢ;
calculating a second homology value y representing a homology between data included in the analysis target data group and the second data group, respectively, wherein the calculating the second homology value y includes setting n thresholds E each indicating a determination criterion for the presence/absence of a homology and calculating the second homology value y as a second homology value yᵢ (i=1, 2, . . . , n) for each threshold Eᵢ; and
determining to which one of the first and second data groups the analysis target data group is similar on the basis of a relationship between the first homology value xᵢ, the second homology value yᵢ, and the number n of thresholds.

13. A homology analysis program product causing a computer system to analyze whether an analysis target data group is similar to a first data group or a second data group wherein the first and second data groups is different from the analysis target data group, comprising:
a recording medium;
a first program code which is recorded on the recording medium and gives the computer system a first command for calculating a first homology value x representing a homology between data included in the analysis target data group and the first data group, respectively, wherein the first command includes setting n thresholds E each indicating a determination criterion for the presence/absence of a homology and calculating a first homology value xᵢ (i=1, 2, . . . , n) for each threshold Eᵢ;
a second program code which is recorded on the recording medium and gives the computer system a second command for calculating a second homology value y representing a homology between data included in the analysis target data group and the second data group, respectively, wherein the second command includes setting n thresholds E each indicating a determination criterion for the presence/absence of a homology and calculating a second homology value yᵢ (i=1, 2, . . . , n) for each threshold Eᵢ; and
a third program code which is recorded on the recording medium and gives the computer system a third command for determining to which one of the first and second data groups the analysis target data group is similar on the basis of a relationship between the first homology value xᵢ, the second homology value yᵢ, and a number n of thresholds.

14. A transaction establishment system for analyzing whether a transaction condition including at least two transaction condition data of a first transaction party is similar to a transaction condition including at least two transaction conditions presented by any one of at least two second transaction parties to determine establishment of a transaction, thereby determining whether a transaction is established between the first transaction party and at least the two second transaction parties, comprising:
a first homology value calculation unit calculating a first homology value x representing a homology between the transaction condition data of the first transaction party and the transaction condition data of one of the second transaction parties, wherein the first homology value calculating unit sets n thresholds E each indicating a determination criterion for the presence/absence of a homology and calculates a first homology value xᵢ (i=1, 2, . . . , n) for each threshold Eᵢ; and
a second homology value calculation unit calculating a second homology value y representing a homology between at least two transaction condition data of the first transaction party and transaction condition data of the other party who is not a target for which the first homology value calculation unit performed homology value calculation, wherein the second homology value calculating unit sets n thresholds E each indicating a determination criterion for the presence/absence of a homology and calculates a second homology value yᵢ (i=1, 2, . . . , n) for each threshold Eᵢ,
wherein the establishment of the transaction is determined on the basis of the first homology value xᵢ and the second homology value yᵢ.

15. A transaction establishment system according to claim 14, further comprising transaction establishment determination unit determining to which the transaction condition presented by any one of the second transaction parties the transaction condition of the first transaction party is similar on the basis of a relationship between the first homology value xᵢ, the second homology value yᵢ, and a number n of thresholds.

16. A transaction establishment system according to claim 14, wherein
one transaction condition data of the first transaction party is made to correspond to one transaction condition data of the second transaction party, and
the first homology value calculation unit and the second homology value calculation unit calculate homology values between transaction condition data which are made to correspond to each other.

17. A transaction establishment system according to claim 15, wherein
one transaction condition data of the first transaction party is made to correspond to one transaction condition data of the second transaction party, and the first homology value calculation unit and the second homology value calculation unit calculate homology values between transaction condition data which are made to correspond to each other.

18. A transaction establishment system according to claim 15, wherein the transaction establishment unit derives in order of similarity the second transaction party who has presented a transaction condition similar to a transaction condition of the first transaction party on the basis of a relationship between the first homology value xᵢ, the second homology value yᵢ, and the number n of thresholds.
